(19) United States
(12) Patent Application Publication
(10) Pub. No.: US 2004/0067248 A1
(43) Pub. Date: Apr. 8, 2004

(54) Animal tag

(76) Inventor: Gillian Mary Mathias, Loughborough (GB)

Correspondence Address: PAUL E MILLIKEN, 9061 WALL STREET, NW, MASSILLON, OH, 44646-1676, US

(21) Appl. No.: 10/659,605

(22) Filed: Sep. 10, 2003

(30) Foreign Application Priority Data
Oct. 4, 2002 (GB) GB 0222985.4

(51) Int. Cl. A61K 35/78

(52) U.S. Cl. 424/422; 424/736; 424/745; 424/747; 424/739; 424/750; 424/769

(57) ABSTRACT

An animal tag comprises a plastics element impregnated with essential oils, with a hole therethrough, enabling attachment of the animal tag to an animal collar. The oils used may be blended. The concentration of essential oils in the plastics is between 10 and 20 percent, but is preferably about 15 percent. The animal tag can be used for example to treat fleas, ticks and other parasites; to keep the coat in good condition; to treat cuts or grazes, bites, stings, irritated skin, allergies, colds and flu; to kill bacteria and viruses on bedding and blankets; to treat arthritis, bad breath and moods.

### FIELD OF THE INVENTION

The present invention relates to an animal tag and more particularly but not exclusively to a pet tag, for example a dog tag.

### BACKGROUND OF THE INVENTION

It is well known that essential oils are used in human treatment. However, until now, it has not been known to use essential oils in the treatment of animals. It has been found that essential oils are particularly useful in treating dogs, and can be used to control fleas, ticks and other parasites; to keep the coat in good condition; to treat cuts or grazes, bites, stings, irritated skin, allergies, colds and flu; to kill bacteria and viruses on bedding and blankets; to treat arthritis and bad breath.

### SUMMARY OF THE INVENTION

According to the present invention there is provided an animal tag comprising a plastics element impregnated with essential oils.

It is an advantage of the invention that the animal cannot ingest the essential oils, since they are contained in the plastics element.

The plastics element may have a hole therethrough enabling attachment of the animal tag to an animal collar.

Preferably the plastics element is a substantially flat circular disc, which when attached to an animal collar with a split ring or other releasable fastening device, allows the animal tag to rest against the animal, close to its nose.

Preferably the plastics element releases minute levels of essential oils into the air as it is heated by the body heat of the animal. The airborne essential oil molecules are picked up through receptor cells in the animal's nose and dissolve into the nasal cavity. Typically a dog's nose has between 125 million and 225 million receptor cells, compared with a human's nose which has only 5 million receptor cells. When a dog salivates, its wet tongue also absorbs scent particles, thus increasing the amount of essential oils absorbed.

The essential oils are released from the plastics element for around 4 weeks, at a concentration of 15 percent essential oils in the plastics.

Preferably the concentration of essential oils in the plastics is between 10 and 20 percent. Most preferably the concentration of essential oils in the plastics is about 15 percent.

The plastics element maybe impregnated with one type of essential oil, or a blend of essential oils, depending on the requirement of the pet being treated.

Preferably Lavender, Sweet Marjoram, Basil, Bitter Orange and Neroli oils are used for relaxing a dog. Rosemary, Rose Geranium, Cinnamon, Coriander and Grapefruit oils maybe used as a pick me up, Peppermint, Lemongrass, and Tea Tree oils for flea and tick control, and Rose, Geranium, Lavender and Spearmint oils as a deodorant.

### DESCRIPTION OF THE DRAWING

The invention will now be described by way of example only with reference to the following drawing in which:

FIG. 1 shows an animal tag attached to the collar of a dog.

### DETAILED DESCRIPTION OF THE INVENTION

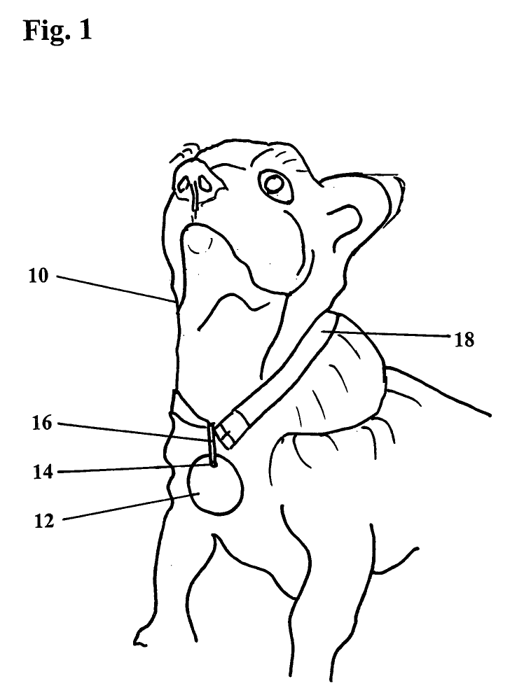

Referring to FIG. 1, a dog 10 is shown wearing an animal tag 12. The animal tag 12 is a plastics element, in the form of a substantially flat circular disc, impregnated with essential oils. An aperture 14 is provided through the disc 12, enabling the disc to be attached to a collar 18 by a split ring 16.

The dog 10 cannot ingest the essential oils, since they are contained in the plastics element 12. Even licking of the plastics element will not release sufficient essential oils to harm the animal by ingestion. A typical dog can ingest up to 5 ml of essential oils at one time without ill effects.

The plastics element releases minute levels of essential oils into the air as it is heated by the body heat of the animal. The airborne essential oil molecules are picked up through receptor cells in the animal's nose and dissolve into the nasal cavity. Typically a dog's nose has between 125 million and 225 million receptor cells, compared with a human's nose which has only 5 million receptor cells. When a dog salivates, its wet tongue also absorbs scent particles, thus increasing the amount of essential oils absorbed.

The concentration of essential oils in the plastics is preferably between 10 and 20 percent, but is most preferably about 15 percent. The essential oils are released from the plastics element 12 for around 4 weeks, at a concentration of 15 percent, once removed from sealed packaging.

The plastics element maybe impregnated with one type of essential oil, or a blend of essential oils, depending on the requirement of the pet being treated.

It has been found that essential oils can be used to help balance the mood of a dog, for example, Lavender, Sweet Marjoram, Basil, Bitter Orange and Neroli oils can be used for relaxing a dog. Rosemary, Rose Geranium, Cinnamon, Coriander and Grapefruit oils can be used as a pick me up, Peppermint, Lemongrass, and Tea Tree oils for flea and tick control, and Rose, Geranium, Lavender and Spearmint oils as a deodorant.

1. An animal tag comprising a plastics element impregnated with essential oils.

2. An animal tag according to claim 1 wherein the plastics element is a substantially flat circular disc.

3. An animal tag according to claim 1 wherein the plastics element has a hole therethrough enabling attachment of the animal tag to an animal collar.

4. An animal tag according to claim 1 wherein the plastics element is attached to an animal collar with a split ring, which allows the animal tag to rest against an animal, close to its nose.

5. An animal tag according to claim 1 wherein the plastics element is impregnated with a blend of essential oils.

6. An animal tag according to claim 1 wherein the essential oils are Lavender, Sweet Marjoram, Basil, Bitter Orange and/or Neroli oils.

7. An animal tag according to claim 1 wherein the essential oils are Rosemary, Rose Geranium, Cinnamon, Coriander and Grapefruit oils.

8. An animal tag according to claim 1 wherein the essential oils are Peppermint, Lemongrass, and Tea Tree oils.

9. An animal tag according to claim 1 wherein the essential oils are Rose, Geranium, Lavender and Spearmint oils

10. An animal tag according to claim 1 wherein the concentration of essential oils in the plastics element is between 10 and 20 percent.

11. An animal tag according to claim 1 wherein the concentration of essential oils in the plastics element is substantially 15 percent.
